(19) United States
(12) Patent Application Publication
(10) Pub. No.: US 2019/0320971 A1
(43) Pub. Date: Oct. 24, 2019

(54) USER TERMINAL, SERVER, IMPROVEMENT PROPOSAL CREATION METHOD, AND STATE DATA GENERATION METHOD

(71) Applicants: OMRON CORPORATION, Kyoto-shi (JP); OMRON HEALTHCARE CO., LTD., Kyoto (JP)

(72) Inventors: Naoki TSUCHIYA, Otsu-shi (JP); Hiroshi USUI, Kyoto (JP); Yoshiyuki MORITA, Ngaokakyo-shi (JP)

(21) Appl. No.: 16/459,692

(22) Filed: Jul. 2, 2019

Related U.S. Application Data
(63) Continuation of application No. PCT/JP2017/044391, filed on Dec. 11, 2017.

(30) Foreign Application Priority Data
Jan. 4, 2017 (JP) 2017-000244

(51) Int. Cl. A61B 5/00; A61B 5/0205; A61B 5/021

(52) U.S. Cl. CPC A61B 5/486; A61B 5/0205; A61B 5/02438; A61B 5/021; A61B 5/7278; A61B 5/4857

(57) ABSTRACT

According to an embodiment, a user terminal includes a processor. The processor is configured to: calculate feature quantities based on vital data, acceleration data, angular velocity data, and/or environmental data to obtain a feature vector including the feature quantities as elements; code at least a part of the elements of the feature vector to generate state data; and transmit the state data. The feature vector includes a first feature quantity as an element. The first feature quantity includes a feature quantity based on vital data, acceleration data, angular velocity data, and/or environmental data in a first time unit.

### CROSS-REFERENCE TO RELATED APPLICATIONS

This application is a Continuation Application of PCT Application No. PCT/JP2017/044391, filed Dec. 11, 2017, the entire contents of which are incorporated herein by reference.

### FIELD

Embodiments described herein relate to a data transmission technique in a user terminal that measures vital information.

### BACKGROUND

It is desirable for patients with abnormal blood pressure (typically high blood pressure) to have their blood pressure managed on a daily basis. Jpn. Pat. Appln. KOKAI Publication No. 2016-27460 discloses a health management system for displaying a user's health condition to be intuitively recognizable. Specifically, a user terminal transmits measurement data to a server apparatus. The server apparatus calculates a health evaluation value and returns an object corresponding to the health evaluation value to the user terminal. Then, the user terminal displays the object.

Conventional stationary blood pressure measurement devices are difficult to carry around. Thus, a user is plagued by a heavy burden when measuring blood pressure at his or her workplace or any other locations outside the home. On top of that, it is extremely difficult to capture sharp blood pressure fluctuation that could lead to a risk of developing a cerebrovascular or cardiovascular disease, with blood pressure measurement performed only several times a day.

In recent years, the development of sensor technology has yielded user terminals that are simply worn on a wrist of a user for example to enable the user to measure his or her blood pressure. Such a user terminal enables the user to timely measure his or her blood pressure while being free of the heavy burden. Such user terminals employ schemes such as tonometry for example to be capable of implementing continuous measurement on a beat-to-beat basis.

Continuous measurement of user's vital information means that a large amount of the user's vital data is generated. For example, since human's daily heart rate is about 100,000, blood pressure data of about 100,000 sets per day will be generated for each user.

In order to entirely store a large amount of vital data, a large capacity storage is required. If a large amount of vital data is to be transmitted to an external device to be accessible from a doctor or a health instructor, a channel established with the external device is heavily loaded and a large amount of power is consumed. Such a problem would be prominent in a potential case that additional data needs to be transmitted in addition to the vital data, to be used by a doctor, a health instructor, or a computer for decision making related to health guidance for the user. The additional data includes acceleration data, angular velocity data, environmental data, and the like.

### BRIEF DESCRIPTION OF THE DRAWINGS

FIG. 1 is a block diagram illustrating a user terminal according to a first embodiment.

FIG. 2 is a diagram illustrating the appearance of the user terminal in FIG. 1.

FIG. 3 is a diagram illustrating a vital information management system including the user terminal in FIG. 1.

FIG. 4 is an explanatory diagram of feature quantities calculated by a feature calculator in FIG. 1.

FIG. 5 is an explanatory diagram of state data generated by feature coder in FIG. 1.

FIG. 6 is a flowchart illustrating operations performed by the user terminal in FIG. 1.

FIG. 7 is a block diagram illustrating a server according to a second embodiment.

FIG. 8 is a flowchart illustrating operations performed by the server in FIG. 7.

### DETAILED DESCRIPTION

A user terminal according to an embodiment includes a feature calculator, a feature coder, and a communicator. The feature calculator calculates a plurality of feature quantities based on at least one of vital data generated by a vital sensor, acceleration data generated by an accelerometer, angular velocity data generated by a gyro sensor, and environmental data generated by an environment sensor to obtain a feature vector including the plurality of feature quantities as elements. The feature coder codes at least a part of the elements of the feature vector to generate first state data. The communicator transmits the first state data. The feature vector includes a first feature quantity as an element. The first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

A server according to an embodiment includes a communicator, a state data storage, a state transition model storage, and a factor analyze. The communicator receives first state data. The state data storage stores received state data including the first state data. The state transition model storage stores a state transition model that models state transition between a plurality of different user states. The factor analyzer analyzes a factor that has led to transition from a past user state, corresponding to second state data received earlier than the first state data, to a current user state, corresponding to the first state data, using the state transition model, to obtain a factor analysis result. The first state data is obtained by coding at least a part of elements of a feature vector including a plurality of feature quantities as elements based on at least one of vital data generated by a vital sensor, acceleration data generated by an accelerometer, angular velocity data generated by a gyro sensor, and environmental data generated by an environment sensor. The feature vector includes a first feature quantity as an element. The first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

A server according to an embodiment includes a communicator, a state data storage, a state transition model storage, and an improvement proposal creator. The communicator receives first state data. The state data storage stores received state data including the first state data. The state transition model storage stores a state transition model that models state transition between a plurality of different user states. The improvement proposal creator creates an improvement proposal for causing transition from a current user state, corresponding to the first state data, to a user state defined as being better, using the state transition model. The first state data is obtained by coding at least a part of elements of a feature vector including a plurality of feature quantities as elements based on at least one of vital data generated by a vital sensor, acceleration data generated by an accelerometer, angular velocity data generated by a gyro sensor, and environmental data generated by an environment sensor. The feature vector includes a first feature quantity as an element. The first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

An improvement proposal creation method according to an embodiment includes receiving state data. The method includes creating an improvement proposal for causing transition from a current user state, corresponding to the state data, to a user state defined as being better, using a state transition model that models state transition between a plurality of different user states. The state data is obtained by coding at least a part of elements of a feature vector including a plurality of feature quantities as elements based on at least one of vital data generated by a vital sensor, acceleration data generated by an accelerometer, angular velocity data generated by a gyro sensor, and environmental data generated by an environment sensor. The feature vector includes a first feature quantity as an element. The first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

A state data generation method according to an embodiment includes calculating a plurality of feature quantities based on at least one of vital data generated by a vital sensor, acceleration data generated by an accelerometer, angular velocity data generated by a gyro sensor, and environmental data generated by an environment sensor to obtain a feature vector including the plurality of feature quantities as elements. The method includes coding at least a part of the elements of the feature vector to generate state data. The feature vector includes a first feature quantity as an element. The first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

According to an embodiment, it is possible to reduce the amount of transmission data from a user terminal to an external device.

Embodiments will be described below with reference to the drawings. In the description below, elements which are the same as or similar to the already described elements are denoted by the same or similar reference numerals, and redundant descriptions will be basically omitted.

### First Embodiment

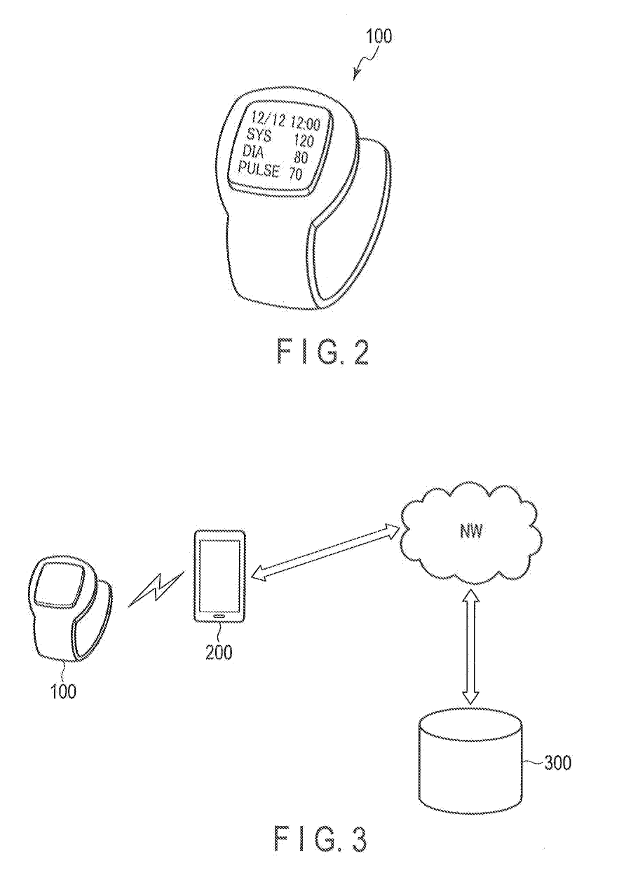

A user terminal according to a first embodiment may be, for example, a watch-type wearable terminal as illustrated in FIG. 2. For example, this user terminal 100 displays information displayed on a general clock such as current date and current time, and further displays vital information about the user such as Systolic Blood Pressure (SYS), Diastolic Blood Pressure (DIA), and pulse rate PULSE. The user terminal 100 can continuously measure the vital information about the user, for example, on a beat-to-beat basis, and display the latest SYS and DIA.

The user terminal 100 may be connected to a smart device (typically, a smartphone or a tablet) 200 as illustrated in FIG. 3. The smart device 200 charts and displays state data transmitted by the user terminal 100, and transmits the state data to a server 300 via a network NW. Details of the state data will be described later. The smart device 200 may have an application installed to manage state data.

The server 300 accumulates the state data transmitted from the user terminal 100 or the smart device 200. The server 300 may transmit the state data of a user to be used for health guidance or diagnosis of the user, in response to access from a Personal Computer (PC) or the like installed in a medical institution, for example.

In addition, as described later, the server 300 analyzes a factor which has led to a change in a user state based on the accumulated state data, or creates an improvement proposal for turning the user state into a user state defined as being better based on the accumulated state data. Then, the factor analysis result and the improvement proposal are transmitted by the server 300 to the user terminal 100 or the smart device 200.

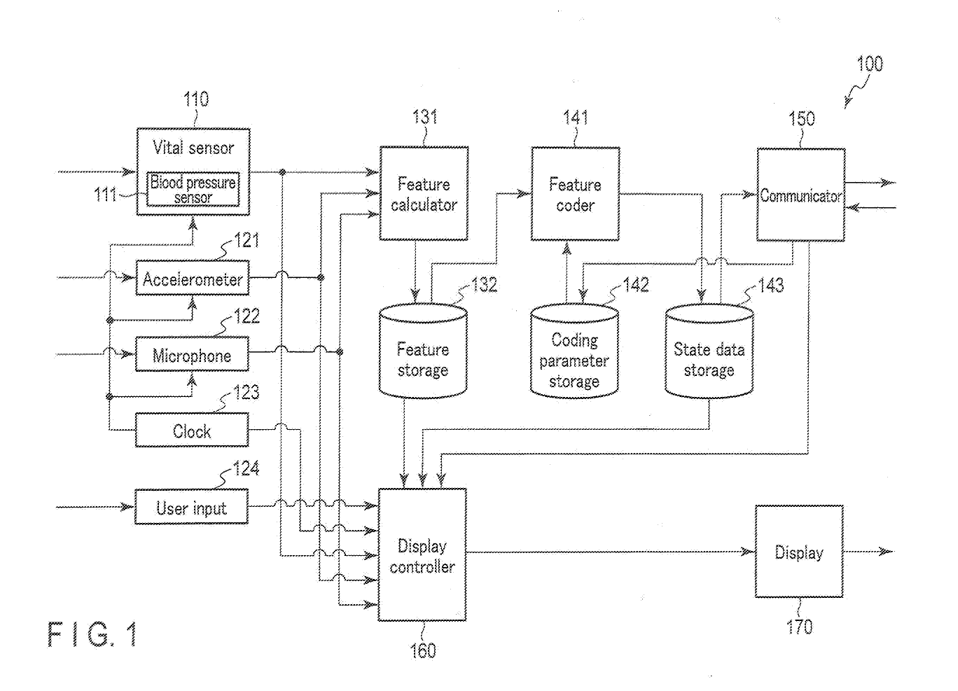

As illustrated in FIG. 1, the user terminal 100 according to the first embodiment includes a vital sensor 110, an accelerometer 121, an environment sensor 122, a clock 123, a user input 124, a feature calculator 131, a feature storage 132, a feature coder 141, a coding parameter storage 142, a state data storage 143, a communicator 150, a display controller 160, and a display 170.

The vital sensor 110 obtains vital data by measuring (for example, continuously measuring) vital information about the user, and transmits the vital data to the feature calculator 131 and the display controller 160. The vital sensor 110 at least includes a blood pressure sensor 111 that obtains blood pressure data by measuring the user's blood pressure. Thus, the vital data at least includes blood pressure data. The blood pressure data may include, for example, but not limited to, systolic blood pressure and diastolic blood pressure values per beat. The vital data can further include electrocardiogram data, heart rate data, pulse wave data, pulse data, body temperature data, and the like. Each vital data can be associated with measurement time set based on time information received from the clock 123.

The blood pressure sensor 111 can include a blood pressure sensor (hereinafter, referred to as a continuous blood pressure sensor) capable of continuously measuring the blood pressure of the user on a beat-to-beat basis. The continuous blood pressure sensor may continuously measure the blood pressure of the user based on Pulse Transit Time (PTT), or may employ tonometry or other techniques to implement the continuous measurement.

The blood pressure sensor 111 can include, in addition to the continuous blood pressure sensor, a blood pressure sensor (hereinafter, referred to as a non-continuous blood pressure sensor) incapable of implementing the continuous measurement. The non-continuous blood pressure sensor measures the user's blood pressure using, for example, a cuff as a pressure sensor (oscillometry).

The non-continuous blood pressure sensor (oscillometric blood pressure sensor in particular) tends to have higher measurement accuracy than continuous blood pressure sensors. In view of this, the blood pressure sensor 111 may measure the blood pressure data with higher accuracy in the following manner, for example. Specifically, the non-continuous blood pressure sensor may be activated to operate instead of the continuous blood pressure sensor, with the activation triggered by satisfaction of a predetermined condition (for example, a condition satisfied when the user's blood pressure data measured by the continuous blood pressure sensor indicates a predetermined high risk state).

The accelerometer 121 detects acceleration received by the accelerometer 121 to obtain three-axis acceleration data. This acceleration data can be used to estimate the activity state (posture and/or action) of the user wearing the user terminal 100. The accelerometer 121 transmits acceleration data to the feature calculator 131 and the display controller 160. The acceleration data may be associated with the measurement time set based on the time information received from the clock 123.

The user terminal 100 may include a gyro sensor instead of or in addition to the accelerometer 121. The gyro sensor detects rotation and obtains angular velocity data. This angular velocity data can be used to estimate the activity state of the user wearing the user terminal 100. The gyro sensor transmits the angular velocity data to the feature calculator 131 and the display controller 160. The angular velocity data may be associated with the measurement time set based on the time information received from the clock 123.

The environment sensor 122 obtains environment data by measuring information about the environment around the user terminal 100, and transmits the environment data to the feature calculator 131 and the display controller 160. The environmental data can include temperature data, humidity data, barometric pressure data, and the like. Each environmental data may be associated with the measurement time set based on the time information received from the clock 123.

The clock 123 generates time information indicating the current time at a predetermined interval, and transmits the time information to the vital sensor 110, the accelerometer 121 (and/or the gyro sensor), the environment sensor 122, and the display controller 160. The time information can be used as the measurement time of vital data obtained by the vital sensor 110, the measurement time of acceleration data obtained by the accelerometer 121 (and/or angular velocity data by the gyro sensor), the measurement time of environmental data obtained by the environment sensor 122, and the like.

The clock 123 may have a calendar function. Thus, the clock 123 may generate date information indicating current date, for example, and transmit the date information to the display controller 160. For example, the date information is useful for the analysis of vital information, because blood pressure may fluctuate differently among days of the week and seasons in addition to the regular daily fluctuation.

The user input 124 is a button, a dial, a crown, or the like for receiving user input. Alternatively, a combination of the user input 124 and the display 170 described later may be implemented using, for example, a touch screen. The user input may be an operation to control the display screen of the display 170 or the like.

The feature calculator 131 receives the vital data from the vital sensor 110, receives the acceleration data from the accelerometer 121 (and/or the angular velocity data from the gyro sensor), and receives the environmental data from the environment sensor 122. The feature calculator 131 calculates a plurality of feature quantities based on the vital data, the acceleration data (and/or angular velocity data), and the environment data, to obtain a feature vector including the plurality of feature quantities as elements. The feature calculator 131 stores the feature vector in the feature storage 132.

The feature vector can include a first feature quantity as an element. The first feature quantity can include at least one of a first vital feature quantity based on vital data in a first time unit, a first activity feature quantity based on acceleration data (and/or angular velocity data) in the first time unit and a first environmental feature quantity based on environmental data in the first time unit. The first time unit may be, for example, one day, one week, one month, one year, or the like.

The feature vector may further include a second feature quantity as an element, in addition to the first feature quantity. The second feature quantity can include at least one of a second vital feature quantity based on vital data in a second time unit longer than the first time unit, a second activity feature quantity based on acceleration data (and/or angular velocity data) in the second time unit and a second environmental feature quantity based on environmental data in the second time unit. The second time unit may be, for example, one week, one month, one year, or the like.

Furthermore, the feature vector can include a third feature quantity as an element, in addition to the first feature quantity and the second feature quantity. The third feature quantity can include at least one of a third vital feature quantity based on vital data in a third time unit longer than the second time unit, a third activity feature quantity based on acceleration data (and/or angular velocity data) in the third time unit and a third environmental feature quantity based on environmental data in the third time unit. The third time unit may be, for example, one month, one year, or the like.

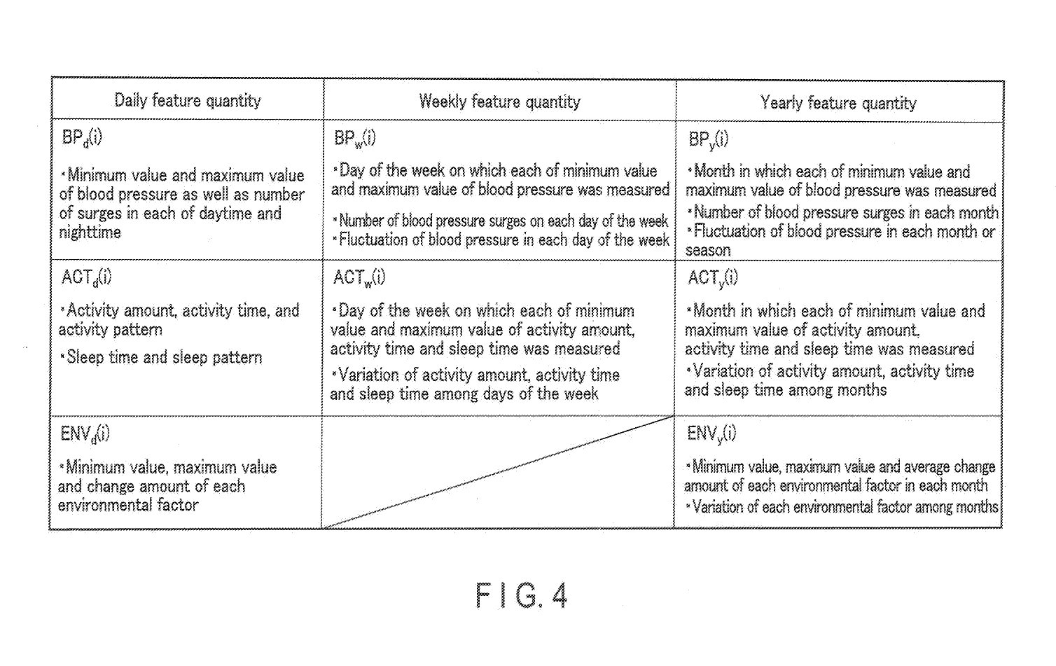

Feature quantities calculated by the feature calculator 131 are illustrated in FIG. 4. In the example illustrated in FIG. 4, the first time unit, the second time unit, and the third time unit are one day, one week, and one year, respectively.

In FIG. 4, BP_{d}(i) represents the first vital feature quantity. The first vital feature quantity illustrated in FIG. 4 includes the minimum value, maximum value and the number of blood pressure surges during the daytime and nighttime on the target day.

The blood pressure surge refers to, for example, sharp blood pressure fluctuation that may be triggered by hypoxia during episodes of Sleep Apnea Syndrome (SAS). Thus, monitoring of the number of blood pressure surges is useful for recognizing the severity of the user's SAS symptom.

Here, i is an integer that is equal to or larger than 1. In the example illustrated in FIG. 4, BP_{d}(1) represents the minimum value of daytime blood pressure on the target day, BP_{d}(2) represents the maximum value of daytime blood pressure on the target day, BP_{d}(3) represents the number of daytime blood pressure surges on the target day, BP_{d}(4) represents the minimum value of nighttime blood pressure on the target day, BP_{d}(5) represents the maximum value of nighttime blood pressure on the target day, and BP_{d}(6) represents the number of nighttime blood pressure surges on the target day.

Since human's daily heart rate is about 100,000, simple collection of all systolic blood pressure data and diastolic blood pressure data per beat results in about 200,000 data pieces. On the other hand, in the example illustrated in FIG. 4, the daily blood pressure behavior can be expressed using six feature quantities. With this conversion of sensor data into feature quantities, the amount of transmission data can be largely reduced compared with a case that the sensor data is directly transmitted.

In FIG. 4, ACT_{d}(i) represents the first activity feature quantity. The first activity feature quantity illustrated in FIG. 4 includes an activity amount, an activity time and an activity pattern on a target day, as well as a sleep time and a sleep pattern on the target day. The first activity feature quantity can be calculated by estimating the activity of the user based on acceleration data (and/or angular velocity data) in the first time unit, using a known technique.

In FIG. 4, ENV_{d}(i) represents the first environmental feature quantity. The first environmental feature quantity illustrated in FIG. 4 includes the minimum value, the maximum value and the amount of change of each environmental factor on the target day. The environmental factor indicates the measurement target of the environment sensor 122, example of which including temperature, humidity, atmospheric pressure, and the like.

In FIG. 4, BP_{w}(i) represents the second vital feature quantity. The second vital feature quantity illustrated in FIG. 4 includes the day of the week on which each of the minimum value and the maximum value of blood pressure was measured in the target week, the number of blood pressure surges on each day of the target week, and the blood pressure fluctuation on each day of the target week.

In FIG. 4, ACT_{w}(i) represents the second activity feature quantity. The second activity feature quantity illustrated in FIG. 4 includes the day of the week on which each of the minimum value and the maximum value of the activity amount, the activity time and sleep time was measured in the target week, as well as variations of the activity amount, the activity time, and the sleep time among the days of the target week.

In the example of FIG. 4, the second environmental feature quantity, that is, the feature quantity based on the environmental data of the target week is not defined. Still, the second environmental feature quantity may be defined and added to the elements of the feature vector. Furthermore, some of the feature quantities illustrated in FIG. 4 may be omitted from the elements of the feature vector.

In FIG. 4, BP_{y}(i) represents the third vital feature quantity. The third vital feature quantity illustrated in FIG. 4 includes the month in which the minimum value and the maximum value of blood pressure were measured in the target year, the number of blood pressure surges in each month in the target year, and the blood pressure fluctuation in each month or season in the target year.

In FIG. 4, ACT_{y}(i) represents the third activity feature quantity. The third activity feature quantity illustrated in FIG. 4 includes the month in the target year in which the minimum values and the maximum values of the activity amount, the activity time and sleep time were measured, as well as variations of the activity amount, the activity time and the sleep time among the months of the target year.

In FIG. 4, ENV_{y}(i) represents the third environmental feature quantity. The third environmental feature quantity illustrated in FIG. 4 includes the minimum value and the maximum value, as well as the average change amount of each environmental factor in each month of the target year, as well as the variation of environmental factor among the months of the target year.

The feature storage 132 stores the feature vector generated by the feature calculator 131. The feature vector (elements of the feature vector) stored in the feature storage 132 is read by the feature coder 141 and the display controller 160 as needed.

The feature coder 141 reads the feature vector from the feature storage 132, and reads the coding parameter from the coding parameter storage 142. The feature coder 141 generates state data by coding each element of the feature vector using the coding parameter. The feature coder 141 stores the state data in the state data storage 143.

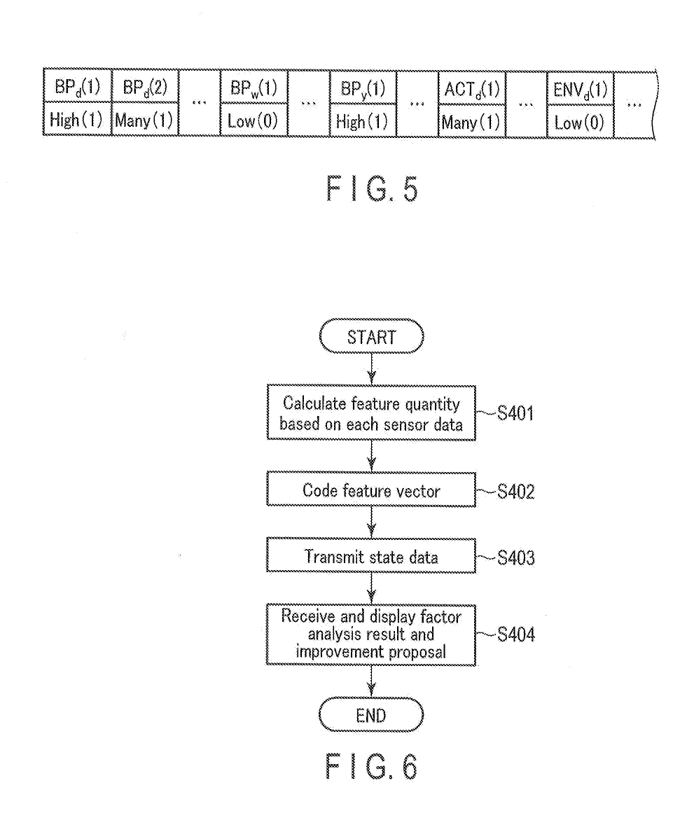

The coding parameters can include one or more thresholds for converting (discretizing) each element of the feature vector into a binary or multi-valued index. For example, in a case that BP_{d}(i) is the maximum value of nighttime blood pressure on the target day, the conversion by the feature coder 141 may result in “1” (high) if BP_{d}(i) is equal to or larger than 130 and result in “0” (low) if BP_{d}(i) is lower than 130. With such coding, BP_{d}(i) can be binarized. By coding (segmenting) each element of the feature vector in this manner, the state data as illustrated in FIG. 5 can be generated for example. Each element of the feature vector is discretized, and thus the data size of the state data is smaller than that of the feature vector. Note that some of the elements of the feature vector may not be coded (may be raw data).

The threshold can be set for each feature quantity. Each threshold may be determined based on, for example, a value defined in a medical system guideline, or may be determined from a statistical distribution of a group of feature quantities. That is, the feature coder 141 may perform the segmentation based on a value defined in the guideline, may perform the segmentation based on correlation in the distribution of existing data, or may perform the segmentation based on existing data and a probability of occurrence of an effect such as depression. The threshold may be set in the coding parameter storage 142 from an external device via the network NW and the communicator 150.

The coding parameter storage 142 stores, for example, a coding parameter including the threshold described above. The coding parameter stored in the coding parameter storage 142 is read by the feature coder 141 as needed. Furthermore, the coding parameters may be updated using the coding parameters received by the communicator 150. Note that a mechanism for updating coding parameters is not an essential feature. In other words, the coding parameter may be set while the user terminal 100 is being manufactured, and may be held statically in the coding parameter storage 142.

The state data storage 143 stores the state data generated by the feature coder 141. The state data stored in the state data storage 143 is read by the communicator 150 and the display controller 160 as needed.

The communicator 150 exchanges data with an external device via the network NW. The communicator 150 may perform one of wireless communications and wired communications or both. For example, the communicator 150 may perform near field communications using Bluetooth (registered trademark) etc. with the smart device 200, for example.

The communicator 150 reads the state data from the state data storage 143, and transmits the state data to the external device. Furthermore, the communicator 150 may receive a coding parameter from the external device, and rewrite the coding parameter stored in the coding parameter storage 142 with the received coding parameter. The communicator 150 may receive the factor analysis result and the improvement proposal to be described later from the external device, and may transmit the factor analysis result and the improvement proposal to the display controller 160.

In the following description, the provision of both the factor analysis result and the improvement proposal is not an essential feature, and only one of or none of these may be provided.

The display controller 160 controls the display 170. Specifically, the display controller 160 generates screen data and sends the screen data to the display 170. The display controller 160 may, for example, generate the screen data based on the vital data from the vital sensor 110, the acceleration data from the accelerometer 121 (and/or angular velocity data from the gyro sensor), the environment data from the environment sensor 122, the time information and date information from the clock 123, the feature quantity from the feature storage 132, the state data from the state data storage 143, the factor analysis result and the improvement proposal from the communicator 150, and the like. The display controller 160 may select information to be used to generate the screen data based on a user input corresponding to an operation of controlling the display screen of the display 170.

When generating the screen data based on state data, the display controller 160 may, for example, rank the user state corresponding to the state data and generate screen data such that whether the rank is high or low can be visually recognized.

The user state may be defined to be in one-to-one correspondence with the state data, but this should not be construed in a limiting sense. For example, a plurality of pieces of different state data may be defined to be associated with a single user state. In this case, the user state may be determined, for example, by a part of state data (for example, an element related to blood pressure). For example, state data pieces, obtained by coding feature vectors with the same vital feature quantity and with different activity feature quantities and environment feature quantities, may be associated with a single user state. In this case, the user state can be referred to as a user's health condition. Elements of the state data not involved in the determination of the user state may be, for example, used in the server 300 for modeling state transition between different user states, for analysis on a factor that has led to a change in a user state, for creating an improvement proposal for improving a user state, and the like.

The display 170 is, for example, a liquid crystal display, an organic electroluminescence (EL) display, or the like. The display 170 can notify the user of various pieces of information by displaying the screen data from the display controller 160. Specifically, the display 170 may display vital information (such as blood pressure, electrocardiogram, heart rate, pulse wave, pulse rate, and body temperature, for example), acceleration data, angular velocity data, activity amount information (such as the number of steps and calorie consumption calculated based on acceleration data and/or angular velocity), sleep information (such as sleep time, for example), environmental information (such as temperature, humidity, and atmospheric pressure, for example), feature vector (elements of the feature vector), state data, factor analysis results, improvement proposal, current time, calendar, and the like.

The user terminal 100 operates as illustrated in FIG. 6. The operations in FIG. 6 may be performed periodically, at an interval matching the first time unit described above, for example.

First of all, the feature calculator 131 calculates a feature quantity based on each sensor data generated by the vital sensor 110, the accelerometer 121 (and/or the gyro sensor), and the environment sensor 122 to obtain a feature vector (step S401).

The feature coder 141 codes the feature vector obtained in step S401 using the coding parameter to generate state data (step S402). The communicator 150 transmits the state data generated in step S402 to an external device via the network NW (step S403).

The state data transmitted in step S403 is received by the server 300 directly or indirectly (for example, via the smart device 200). The server 300 analyzes the factor that has led to a change in the user state, or creates an improvement proposal for turning a user state into a user state defined as being better. The communicator 150 receives the factor analysis result and the improvement proposal, and the display 170 displays them (step S404).

As described above, the user terminal according to the first embodiment calculates feature quantities based on sensor data in a predetermined time unit, generates state data by coding these feature quantities, and transmits the state data to an external device. Thus, this user terminal can drastically reduce the amount of transmission data, from that in a case where the sensor data is entirely transmitted to an external device such as a smart device or a server. Thus, reduction in power consumption and the load on the channel, involved in the transmission of sensor data, can be achieved. Furthermore, with the state data accumulated instead of the sensor data, reduction in the capacity of the storage (state data storage) can further be achieved. Furthermore, the user terminal displays the user state corresponding to the state data, and displays the factor analysis result and the improvement proposal provided based on the state data that has been transmitted. Thus, this user terminal can encourage the user to change his or her behavior.

### Second Embodiment

As described above, the server to which the state data has been transmitted from the user terminal according to the first embodiment can analyze a factor that has led to a change in a user state, or create an improvement proposal for turning the user state into a user state defined as being better. A second embodiment relates to such a server.

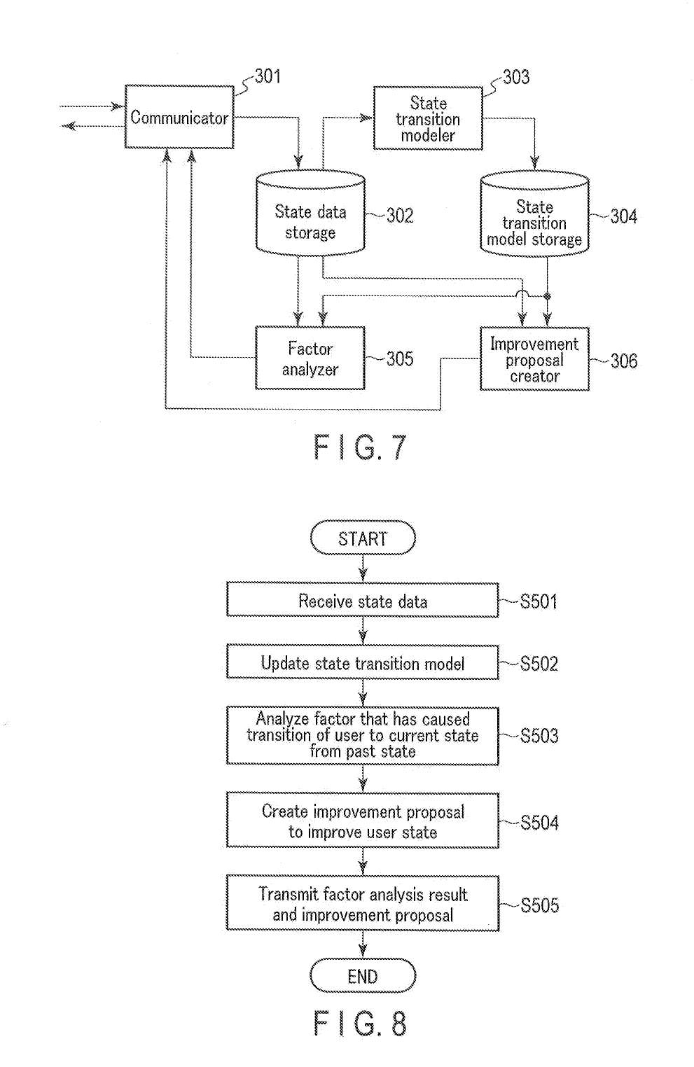

As illustrated in FIG. 7, the server 300 according to the second embodiment includes a communicator 301, a state data storage 302, a state transition modeler 303, a state transition model storage 304, a factor analyzer 305, and an improvement proposal creator 306.

The communicator 301 receives state data from the user terminal 100 via the network NW. An identifier of (the user of) the user terminal 100 that has transmitted the state data may be added to the state data. The server 300 can manage the state data for each user using such an identifier. The communicator 301 stores the received state data in the state data storage 302 (in association with the identifier).

The communicator 301 receives the factor analysis result from the factor analyzer 305, and receives the improvement proposal from the improvement proposal creator 306. The communicator 301 transmits the factor analysis result and the improvement proposal to the user terminal 100 or the smart device 200 via the network.

The state data storage 302 stores state data. In the state data storage 302, for example, a database for managing state data for each user is established. The state data accumulated in the state data storage 302 can be used to analyze the temporal change of the user state. The state data stored in the state data storage 302 is read by the state transition modeler 303, the factor analyzer 305, and the improvement proposal creator 306 as needed.

The state transition modeler 303 reads the state data from the state data storage 302. The state transition modeler 303 models state transition between a plurality of different user states based on the state data. The state transition modeler 303 stores the generated state transition model in the state transition model storage 304.

The state transition model can include, for example, a probability of state transition (conditional probability) related to transition from each user state to another user state. The probability of state transition may be, for example, a probability P_{T} (sₜ₊₁|sₜ, fₜ) of transitioning from a user state sₜ to a user state sₜ₊₁ when a factor fₜ occurs The factor may include an internal factor (an action taken by the user, for example) and an external factor (an environment in which the user is placed, for example). Furthermore, a static parameter such as the user's age group and gender may be added to the condition.

The state transition model may be dynamically changed by the state transition modeler 303, or may be static. When a static state transition model is used, the state transition modeler 303 may be omitted from the server 300.

The state transition model storage 304 stores a state transition model. The state transition model stored in the state transition model storage 304 is read by the factor analyzer 305 and the improvement proposal creator 306 as needed.

The factor analyzer 305 reads current state data and past state data of the target user from the state data storage 302, and reads the state transition model from the state transition model storage 304. The current state data may be state data the date of storage in the state data storage 302 of which is the earliest (for example, the today), and the past state data may be state data the date of storage in the state data storage 302 of which is the second earliest (for example, the yesterday).

The factor analyzer 305 analyzes the factor that has led to transition from a past user state sₜ₋₁, corresponding to the past state data, to the current user state sₜ, corresponding to the current state data, using the state transition model. For example, the factor analyzer 305 may estimate a factor fₜ₋₁ (for example, a low highest temperature) involving the highest probability of state transition PT (sₜ|sₜ₋₁,fₜ₋₁) from the past user state sₜ₋₁ to the current user state sₜ, as the main factor of the state transition, for example. The factor analyzer 305 transmits the factor analysis result (for example, the main factor of the state transition) to the communicator 301. When the factor analysis result is not to be provided, the factor analyzer 305 may be omitted from the server 300.

The improvement proposal creator 306 reads the current state data of the target user from the state data storage 302, and reads the state transition model from the state transition model storage 304. The current state data may be state data the date of storage in the state data storage 302 of which is the earliest (for example, the today).

The improvement proposal creator 306 uses the state transition model to create an improvement proposal for causing transition from the current user state sₜ, corresponding to the current state data, to a user state s_{b} defined as being better. The user state s_{b} defined as being better may be, for example, the user state defined to be at the highest rank, or any user state defined to be at a higher rank than the current user state sₜ. The improvement proposal creator 306 may create, as the improvement proposal, information indicating a factor fₜ (an increase in the activity amount for example) involving the highest probability of state transition P_{T}(s_{b}|sₜ,fₜ) from the current user state sₜ to the current user state s_{b} defined as being better. The improvement proposal creator 306 transmits the improvement proposal to the communicator 301. Note that, when the improvement proposal is not to be provided, the improvement proposal creator 306 may be omitted from the server 300.
The improvement proposal creator 306 may look up the improvement proposal from a table described in IF-THEN rules as used in an expert system for example.
The improvement proposal creator 306 may look up the improvement proposal each time using an (advanced) case base such as Watson, for example, associated with the user state.
The improvement proposal creator 306 may evaluate a success probability for each improvement proposal candidate based on results of intervention (provision of improvement proposal) and the user's reactions collected through implementation of one or both of the above two examples. Furthermore, the improvement proposals provided to the user may be narrowed down using this success probability as an index.

The server 300 operates as illustrated in FIG. 8.

First of all, the communicator 301 receives state data generated by any of the user terminals 100 via the network NW (step S501). This state data is stored, for example, in the state data storage 302 in association with the aforementioned identifier.

The state transition modeler 303 may update the state transition model using the state data received in step S501 (step S502). For example, the state transition modeler 303 may adjust the probability of state transition to the user state corresponding to the state data.

Step S502 is optional and can be omitted, for example, when the state transition model is static. Also, step S502 may be performed after step S503 and step S504 described later.

The factor analyzer 305 analyzes the factor that has led to the transition from the past user state, corresponding to the past state data, to the current user state, corresponding to the state data received in step S501, using the state transition model (step S503).

Meanwhile, the improvement proposal creator 306 creates, using the state transition model, an improvement proposal for causing the transition from the current user state, corresponding to the state data received in step S501, to a user state defined as being better (step S504).

Note that steps S503 and S504 may be performed in an order that is reversed from that in FIG. 8 or may be performed in parallel. Furthermore, step S503 can be omitted if the factor analysis result is not to be provided, and step S504 can be omitted if the improvement proposal is not to be provided.

The communicator 301 transmits the factor analysis result obtained in step S503 and the improvement proposal created in step S504 to the user terminal 100 or the smart device 200 via the network NW (step S505).

As described above, the server according to the second embodiment performs factor analysis and creates an improvement proposal using the state transition model for the received state data, and transmits the factor analysis result and improvement proposal to a user terminal or a smart device. Thus, this server can encourage the user to change his or her behavior. The state data received by the server may be the same as the state data described in the first embodiment described above. Thus, reduction in power consumption and the load on the channel involved in the reception of sensor data can be achieved. Furthermore, with the state data accumulated instead of the sensor data, reduction in the capacity of the storage (state data storage) can further be achieved.

The embodiments described above are merely illustrative examples to assist in understanding the inventive concept, and are not intended to limit the scope of the present invention. Various components can be added to, deleted from, or converted in the embodiments without departing from the gist of the present invention.

The various functional units described in the above embodiments may be implemented with a circuit. The circuit may be a dedicated circuit that implements a specific function, or may be a general-purpose circuit such as a processor that is connected to a memory and executes a predetermined program stored in the memory.

At least a part of the processing in each of the above-described embodiments can also be implemented with a general-purpose computer serving as basic hardware. The program for realizing the above process may be stored in a computer readable recording medium to be provided. The program is stored in the recording medium as a file in an installable format or as a file in an executable format. The recording medium includes a magnetic disk, an optical disk (such as CD-ROM, CD-R, or DVD), a magneto-optical disk (such as MO), a semiconductor memory, and the like. The recording medium may be any medium that can store the program to be readable by a computer. Furthermore, a program for implementing the processing described above may be stored on a computer (server) connected to a network such as the Internet, and may be downloaded to a computer (client) via the network.

1. A user terminal comprising:
a memory;
a processor connected to the memory, wherein the processor is configured to:
calculate a plurality of feature quantities based on at least one of vital data generated by a vital sensor, acceleration data generated by an accelerometer, angular velocity data generated by a gyro sensor, and environmental data generated by an environment sensor to obtain a feature vector including the plurality of feature quantities as elements;
code at least a part of the elements of the feature vector to generate first state data; and
transmit the first state data, wherein
the feature vector includes a first feature quantity as an element, and
the first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

2. The user terminal according to claim 1, wherein
the feature vector further includes a second feature quantity as an element, and
the second feature quantity includes at least one of a second vital feature quantity based on vital data in a second time unit longer than the first time unit, a second activity feature quantity based on at least one of acceleration data and acceleration data in the second time unit, and a second environmental feature quantity based on environment data in the second time unit.

3. The user terminal according to claim 1, wherein
the first time unit is one day, and
the first vital feature quantity includes at least one of a minimum value, a maximum value, and number of surges of blood pressure during daytime and nighttime on a target day.

4. The user terminal according to claim 1, wherein the processor is further configured to receive at least one of an analysis result of a factor that has led to transition from a past user state, corresponding to second state data transmitted earlier than the first state data, to a current user state, corresponding to the first state data, and an improvement proposal for causing transition from the current user state to a user state defined as being better.

5. A server comprising:
a memory; and
a processor connected to the memory, wherein the processor is configured to receive first state data;
the memory stores received state data including the first state data, and a state transition model that models state transition between a plurality of different user states,
the processor is further configured to analyze a factor that has led to transition from a past user state, corresponding to second state data received earlier than the first state data, to a current user state, corresponding to the first state data, using the state transition model, to obtain a factor analysis result,
the first state data is obtained by coding at least a part of elements of a feature vector including a plurality of feature quantities as elements based on at least one of vital data generated by a vital sensor, acceleration data generated by an accelerometer, angular velocity data generated by a gyro sensor, and environmental data generated by an environment sensor,
the feature vector includes a first feature quantity as an element, and
the first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

6. A server comprising:
a memory; and
a processor connected to the memory, wherein the processor is configured to receive first state data,
the memory stores received state data including the first state data, and a state transition model that models state transition between a plurality of different user states,
the processor is further configured to create an improvement proposal for causing transition from a current user state, corresponding to the first state data, to a user state defined as being better, using the state transition model,
the first state data is obtained by coding at least a part of elements of a feature vector including a plurality of feature quantities as elements based on at least one of vital data generated by a vital sensor, acceleration data generated by an accelerometer, angular velocity data generated by a gyro sensor, and environmental data generated by an environment sensor,
the feature vector includes a first feature quantity as an element, and
the first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

7. The server according to claim 5, wherein the processor is further configured to generate the state transition model based on the state data stored in the state data storage.

8. An improvement proposal creation method comprising:
receiving, by a processor, state data; and
creating, by the processor, an improvement proposal for causing transition from a current user state, corresponding to the state data, to a user state defined as being better, using a state transition model that models state transition between a plurality of different user states, wherein
the state data is obtained by coding at least a part of elements of a feature vector including a plurality of feature quantities as elements based on at least one of vital data generated by a vital sensor, acceleration data generated by an accelerometer, angular velocity data generated by a gyro sensor, and environmental data generated by an environment sensor,
the feature vector includes a first feature quantity as an element, and
the first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.

9. A state data transmission method comprising:
calculating, by a processor, a plurality of feature quantities based on at least one of vital data generated by a vital sensor, acceleration data generated by an accelerometer, angular velocity data generated by a gyro sensor, and environmental data generated by an environment sensor and obtaining a feature vector including the plurality of feature quantities as elements;
coding, by the processor, at least a part of the elements of the feature vector to generate state data; and
transmitting, by the processor, the first state data from a user terminal to an external apparatus, wherein
the feature vector includes a first feature quantity as an element, and
the first feature quantity includes at least one of a first vital feature quantity based on vital data in a first time unit, a first activity feature quantity based on at least one of acceleration data and angular velocity data in the first time unit, and a first environmental feature quantity based on environmental data in the first time unit.
